(19) United States
(12) Patent Application Publication
(10) Pub. No.: US 2016/0000721 A1
(43) Pub. Date: Jan. 7, 2016

(54) GASTRO-RETENTIVE FORMULATIONS

(71) Applicant: MERCK SHARP & DOHME CORP., Rahway, NJ (US)

(72) Inventors: Pranav Gupta, Springfield, NJ (US); Ashlesh Sheth, Basking Ridge, NJ (US); Ronald L. Smith, Summit, NJ (US)

(73) Assignee: Merck Sharp & Dohme Corp., Rahway, NJ (US)

(21) Appl. No.: 14/653,600

(22) PCT Filed: Dec. 18, 2013
(86) PCT No.: PCT/US13/75929

§ 371 Date: Jun. 18, 2015

Related U.S. Application Data
(60) Provisional application No. 61/740,884, filed on Dec. 21, 2012.

(51) Int. Cl. A61K 9/20; A61K 9/00; A61K 45/06; A61K 31/506

(52) U.S. Cl. CPC A61K 9/2086; A61K 31/506; A61K 9/0065; A61K 9/2031; A61K 45/06

(57) ABSTRACT

The present invention relates to pharmaceutical compositions of the poorly soluble drugs, and pharmaceutically acceptable salts thereof, in a controlled-release gastric retained oral dosage form. Such compositions are formulated so as to deliver the majority of the incorporated drug into the stomach and upper gastrointestinal tract, with restricted drug delivery in the lower gastrointestinal tract. The dosage forms have multiple layers including an active layer with a first swellable polymer with raltegravir incorporated therein and a non-active layer with a second swellable polymer having a similar molecular weight or a higher molecular weight as the swellable polymer in the active layer.

### FIELD OF THE INVENTION

The present invention relates to pharmaceutical formulations of poorly soluble drugs having a narrow gastrointestinal absorption window, and pharmaceutically acceptable salts thereof, in a controlled-release gastric retained oral dosage form. Such formulations are designed to deliver the majority of the incorporated drug into the stomach and upper gastrointestinal tract, with restricted drug delivery in the lower gastrointestinal tract. The present invention also relates to dosage forms that provide for release of poorly soluble drugs, such as raltegravir, in the gastrointestinal tract at an initial ascending absorption rate beginning at about 0 hours to 6 hours and a second ascending absorption rate beginning at about 8, 10, 12 or 15 hours thereby maintaining drug concentration at the desired therapeutic plasma levels for an extended period of time.

### BACKGROUND OF THE INVENTION

Conventional means for delivering drugs are often severely limited by biological, chemical, and physical barriers. Typically, these barriers are imposed by the environment through which delivery occurs, the environment of the target for delivery, and/or the target itself. These barriers are of particular significance in the design of oral delivery systems. Oral delivery of many drugs often requires greater amounts of drug to be administered than if the drug were administered by a different route. Biological and chemical barriers include, but are not limited to, pH variations in the gastrointestinal (GI) tract, stability in the GI tract and metabolism. Physical barriers include, but are not limited to, lipid bi-layers and various organ membranes that are relatively impermeable to certain drugs but must be traversed before reaching a target, such as the circulatory system.

In addition to these physical barriers, there are barriers with regard to site of drug absorption, i.e., their preferential absorption region. See Davis, 2005, Drug Dis Today 10:249-257. Certain drugs may be preferentially absorbed only in the small intestine and the passage of drug through this area is generally complete within three to five hours, regardless of particle size, dosage form (e.g. liquid, microencapsulated) or presence of food. Once such drugs pass their absorption window, very little or no drug absorption takes place in the lower region of the GI tract. This transit time may provide a window of opportunity that is too short to facilitate the adequate absorption of therapeutic quantities of a drug. Such drugs require administration of frequent doses, an inconvenience and expense to patients and clinicians, and which often results in non-compliance by the patient and failure of therapy.

Sustained release dosage forms for oral administration, designed to deliver a pharmacologically active agent over an extended time period, are well known. In particular, dosage forms that are capable of delivering drug to the stomach and gastrointestinal tract in a controlled-release manner are described in U.S. Pat. Nos. 5,007,790; 5,582,837; and 5,972,389. The dosage forms described in these patents utilize a hydrophilic, water-swellable polymer with the drug dispersed therein. The polymeric particles in which the drug is dispersed absorb water, causing the dosage form to swell, which in turn promotes their retention in the stomach and also allows the drug contained in the polymer to dissolve and then diffuse out of the dosage form. For poorly soluble drugs, the release of drug is usually mediated via a result of polymer erosion, i.e., via degradation of the polymeric matrix. Additional controlled-release dosage forms are described in International Patent Application Publication Nos. WO 98/55107 and WO 96/26718. Each of the dosage forms described in these publications is generally applicable only to highly soluble drug agents and would not be expected to be effective for drugs which exhibit poor solubility at low pH.

Controlled release dosage forms for poorly soluble diuretic drugs have been described in U.S. Patent Application Publication No. 20030152622.

### SUMMARY OF THE INVENTION

The present invention provides a multilayer oral dosage form comprising (a) an active (or drug containing) layer that comprises a poorly soluble drug having a narrow absorption window, or a pharmaceutically acceptable salt thereof, and a first swellable polymer with an average molecular weight (M.W.) in the range of 2 million to 5 million and (b) a non-active layer comprising a second swellable polymer with an average molecular weight greater than 4 million, wherein the average molecular weight of the first swellable polymer is less than or equal to the average molecular weight of the second swellable polymer. In an embodiment of the invention, the average molecular weight of the first swellable polymer is less than the average molecular weight of the second swellable polymer. In certain aspects of this embodiment, the average molecular weight of the first swellable polymer is at least 250,000, at least 500,000, or about 1 million less than the average molecular weight of the second swellable polymer. The dosage form is generally in the form of a tablet.

In certain embodiments, the first swellable polymer has an average molecular weight in the range of 2.5 million to 4 million. In one aspect of this embodiment, the first swellable polymer has an average molecular weight of 4 million, which can be a nonionic, water-soluble poly(ethylene oxide) polymer, for example, POLYOX™ WSR-301 (Dow Chemical). In another aspect of this embodiment, the first swellable polymer is present in the active layer at a concentration range from about 5% to about 70%, about 5% to about 50%, or about 5% to about 35%. In certain embodiments, the first swellable polymer is present in the active layer at a concentration range from about 10% to about 70%, about 10% to about 50% or about 10% to about 35%.

In certain embodiments, the second swellable layer has an average molecular weight of 5 million to 10 million. In one aspect of this embodiment, the second swellable polymer has an average molecular weight of 5 million, which can be a nonionic, water-soluble poly(ethylene oxide) polymer, for example, POLYOX™ WSR Coagulant (Dow Chemical). In certain aspects of this embodiment, the second swellable polymer is present in the non active layer at a concentration greater than about 30% w/w, greater than about 50% w/w, greater than about 70% w/w, greater than about 80% w/w, greater than about 90% w/w, greater than about 95% w/w, or greater than about 98% w/w.

In addition to the polymers and drug, if present, the dosage form may further comprise, in one or more layers, a lubricant, disintegrant, filler, surfactant, or any combination thereof. In an embodiment, the lubricant may be selected from magnesium stearate, calcium stearate, stearic acid, sodium stearyl fumarate or a mixture thereof. In an embodiment, the disintegrant may be selected from croscarmellose or crospovidone. In an embodiment, the filler is generally microcrystalline cellulose or lactose. In an embodiment, the surfactant is poloxamer 188 (PLURONIC® F68) or sodium lauryl sulfate.

In certain embodiments, the dosage form provides for retention in the stomach and/or upper gastrointestinal tract for at least 10 hours in a subject in the fed state.

In certain embodiments of the invention, the dosage form further comprises an intermediate release layer (thereby forming a trilayer) comprising either the same drug, or pharmaceutically acceptable salt thereof, as in the first swellable active layer, or a second drug, or pharmaceutically acceptable salt thereof, or combinations of both drugs. In one aspect, the filler comprises cellulose or lactose, and may further comprise magnesium stearate, sodium stearyl fumarate or a mixture thereof, and optionally, a disintegrant or a surfactant. In certain aspects of this embodiment, the second swellable polymer is present in the non-active layer at a concentration greater than about 50% w/w.

In certain embodiments, the poorly soluble drug is raltegravir, which is preferably present as a potassium salt, and more preferably the anhydrous crystalline potassium salt of raltegravir, which is characterized by an X-ray powder diffraction pattern obtained using copper K_{α} radiation which comprises 2Θ values in degrees of 5.9, 20.0 and 20.6. In certain aspects of this embodiment, the multilayer dosage forms (either bilayer or trilayer) may further comprise one or more additional anti-HIV agents.

The present invention also relates to methods for inhibiting HIV integrase, or for the treatment or prophylaxis of HIV infection or the treatment, prophylaxis or delay in the onset of AIDS, in a subject in need of such inhibition which comprises admininstering a dosage form of the invention.

The present invention also relates to dosage forms of the invention for use in the inhibition of HIV integrase, the treatment or prophylaxis of HIV infection, or the treatment, prophylaxis or delay in the onset of AIDS.

The present invention also provides a dosage form comprising a poorly soluble drug, such as raltegravir, that provides a release of the drug in the gastrointestinal tract in a subject under fed conditions with an initial peak within 6 hours post dose and a second peak within 8-24 hours post dose. In certain embodiments, the dosage form provides an initial ascending absorption rate beginning at about 0 hours to 4 hours or beginning at about 0 hours to 6 hours. In certain embodiments, the initial ascending rate is maintained for at least two hours. In certain embodiments, the dosage form provides a second ascending absorption rate in a time period from about 8, 10, 12, 15 hours to about 20 hours. In certain embodiments, the second ascending rate is maintained for at least two hours.

The present invention also relates to a method for treating or preventing HIV infection comprising administering a dosage form comprising raltegravir that provides a release of raltegravir in the gastrointestinal tract in a subject under fed conditions with an initial peak within 6 hours post dose and a second peak within 8-24 hours post dose. In certain embodiments, the dosage form also provides an initial ascending absorption rate beginning at about 0 hours to 4 hours or beginning at about 0 hours to 6 hours. In certain embodiments, the initial ascending rate is maintained for at least two hours. In certain embodiments, the dosage form provides a second ascending absorption rate in a time period from about 8, 10, 12, 15 hours to about 20 hours. In certain embodiments, the second ascending rate is maintained for at least two hours.

Other embodiments, aspects and features of the present invention are either further described in or will be apparent from the ensuing description, examples and appended claims.

### BRIEF DESCRIPTION OF THE DRAWINGS

FIG. 1A represents the drug release profile from the monolithic gastro-retentive formulations (400 mg dose strength) listed in Table 4 in 900 ml of distilled water using the USP I basket method (#20 mesh basket; 100 rpm). FIG. 1B represents the drug release profile from the bilayer gastro-retentive formulations listed in Table 3 in 900 ml of distilled water using the USP I basket method (#20 mesh basket; 100 rpm). FIG. 1C represents the drug release profile from the tri-layered gastro-retentive formulations (750 mg dose strength-150 mg IR/600 mg CR) in 900 ml of distilled water using the USP I basket method (#20 mesh basket; 100 rpm).

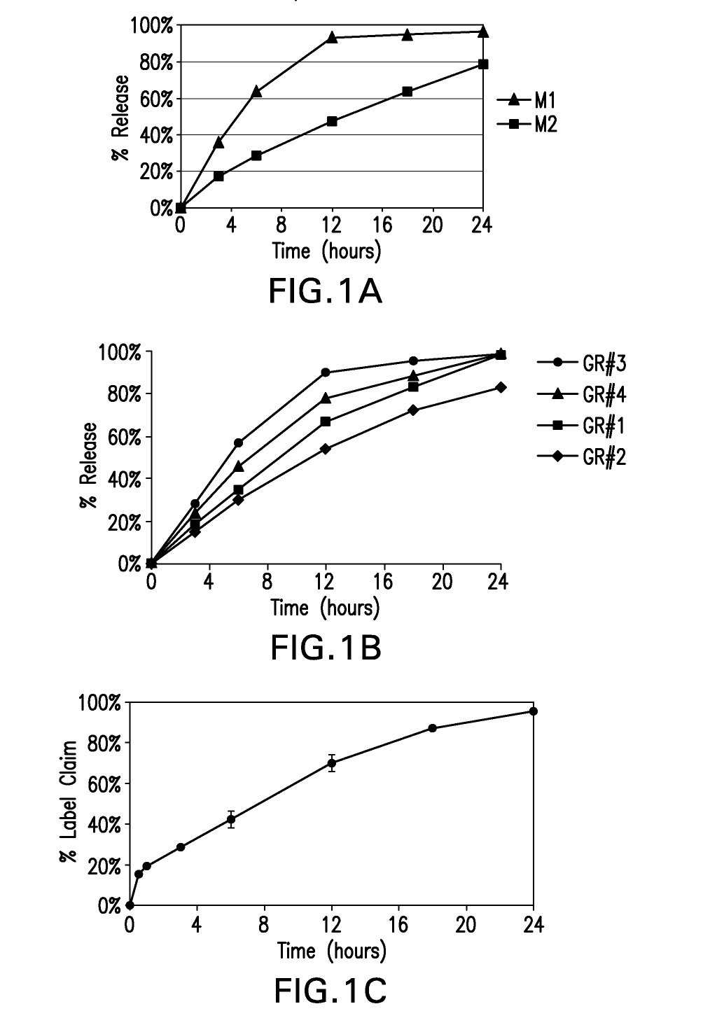

FIG. 2 is the mean raltegravir plasma concentration vs. time profile of raltegravir (Log₁₀/Linear Scale) on day 5 for bi-layered (Regimen B) and tri-layered (Regimen A) GR formulations administered once daily at an oral dose of 1200 mg or 1500 mg respectively for 5 days under fed conditions to healthy subjects in a multi-dose PK study. The mean plasma concentrations at 24 hr time point are at or above 100 ng/ml (44 nM) and shall decline below these conc. levels at time points beyond 24 hours.

### DETAILED DESCRIPTION OF THE INVENTION

The present invention relates to multi-layered dosage forms, e.g., bilayered and trilayered forms, for poorly soluble drugs such as raltegravir, to achieve prolonged gastro-retention while facilitating adequate drug release from the dosage form. In one embodiment, these multi-layered dosage forms of the invention provide for once-daily delivery of raltegravir by means of a gastric retained dosage form for the inhibition of HIV integrase and/or the treatment or prevention of AIDS.

The present invention also relates to 1) dosage forms comprising a poorly soluble drug, such as raltegravir, that provides a release of the drug in the gastrointestinal tract in a subject under fed conditions with ascending absorption rate in a time beginning between about 0 hours to about 6 hours, or between about 1 hours to about 5 hours or that provides a release of the drug in the gastrointestinal tract with an initial peak within 6 hours post dose and a second peak within 8 hours about to 24 hours post dose; and 2) methods for treating or preventing HIV infection comprising administering a dosage form comprising raltegravir that provides a release of raltegravir in the gastrointestinal tract with an ascending absorption rate in a time beginning between about 0 hours to about 6 hours, or between about 0 zero to about 5 hours, or that provides a release of the drug in the gastrointestinal tract with an initial peak within 6 hrs post dose and a second peak within 8-24 hours post dose. In certain embodiments, there is an initial ascending absorption rate beginning at about 0 to 4 hours or beginning at about 0 to 6 hours. In some embodiments, the ascending rate for either the first or second ascending rate can last for at least 1, 2, 3, 4 or 5 hours. In some embodiments, the ascending rate lasts no more than 2, 3, 4, 5, 6, 7, or 8 hours. Various monolayer, bilayer and trilayer formulations of raltegravir showed an unexpected two peak or twin maxima drug absorption profile. As with any drug pharmacokinetic profile, an initial ascending rate was observed followed by a decrease. For raltegravir dosage forms, a second ascending rate was observed beginning at about 8 hours, about 10 hours, about 12 hours, or about 15 hours and lasting until about 20 hours. In certain embodiments, there is a second ascending absorption rate beginning at about 8 hours to about 20 hours or beginning at about 12 to about 20 hours. In some embodiments, the ascending rate lasts no more than 2, 3, 4, 5, 6, 7, or 8 hours. This may correspond to the gastric emptying time resulting from the disintegration of the dosage form as is also indicated by scintigraphic studies. This results in an extended release profile that provides the required therapeutic plasma levels for once daily dosing.

All numbers disclosed herein can be in the form of ranges, for example, for a given number, ±1%, ±2%, ±5%, ±10%, ±15% and ±20% are contemplated. The equivalent numbers represented by these percentages are also contemplated.

All references to “molecular weight”, unless otherwise specified, refer to an average molecular weight.

An “ascending release rate” refers to a periodic release rate that is increased over the immediately-preceding periodic release rate, where the periodic intervals are the same. For example, when the quantity of drug released from a dosage form is measured at hourly intervals and the quantity of drug released during the ninth hour following administration (determined at t=9 hours) is greater than the quantity of drug released from the dosage form during the eighth hour following administration (determined at t=8 hours), an ascending release rate from the eighth hour to the ninth hour has occurred. When an ascending release rate is defined as occurring within a time range, it means that at some point within that range the release is ascending.

It will be appreciated that the first periodic release rate measured, e.g., the periodic release rate at t=1 hour (unless equal to 0), will always be greater than the release rate during the preceding period, e.g., the hour before the dosage form was administered, and, thus, the first periodic release rate always constitutes an occurrence of an ascending release rate.

As used herein, “absorption window” refers to a specific segment of the gastrointestinal tract where a particular drug is absorped. The ability of a drug to be absorbed in a particular segment is related to drug solubility and stability in the particular microenvironment which is dependent on pH, the lipohilicity and intrinsic membrane permeability of the drug, the presence of drug transport mechanisms and the like.

As used herein, the phrase “controlled release” refers to any drug-containing formulation in which release of the drug is not immediate, i.e., with a “controlled release” formulation, oral administration does not result in immediate release of the drug into an absorption pool.

As used herein, the phrase “dosage form” refers to any form of a pharmaceutical composition that contains an amount of drug, or a pharmaceutically acceptable salt thereof, sufficient to achieve a therapeutic effect with a single administration. When the formulation is a tablet, the dosage form is usually one such tablet but can be two or more. The frequency of administration that will provide the most effective results in an efficient manner without overdosing will vary with: (1) the characteristics of drug, including both its pharmacological characteristics and its physical characteristics, such as solubility and lipophilicity; (2) the characteristics of the swellable matrix, such as its diffusion permeability; and (3) the relative amounts of the drug and polymer. In most cases, the dosage form will be such that effective results will be achieved with administration no more frequently than once every twelve hours or more, and preferably once every twenty-four hours or more.

As used herein, the term “drug”, “active agent,” and “pharmacologically active agent” are used interchangeably herein to refer to any chemical compound, complex or composition that is suitable for oral administration and that has a beneficial biological effect, preferably a therapeutic effect in the treatment of a disease or abnormal physiological condition. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent” and “drug” are used, then, or when a particular active agent is specifically identified, it is to be understood that applicants intend to include the active agent per se as well as pharmaceutically acceptable, pharmacologically active salts, esters, amides, prodrugs, metabolites, analogs, etc. More specifically, the terms “active agent,” “pharmacologically active agent,” and “drug” are intended to include the poorly soluble drugs with narrow gastrointestinal absorption windows to which this invention is directed. Drug compositions are generally utilized clinically in the form of a pharmaceutically acceptable salt thereof. Accordingly, the term “drug” refers to a clinically useful form of a drug composition including a pharmaceutically acceptable salt thereof.

As used herein, the term “fed mode,” refers to a state which is typically induced in a patient by the presence of food in the stomach, the food giving rise to two signals, one that is said to stem from stomach distension and the other a chemical signal based on food in the stomach. It has been determined that once the fed mode has been induced, larger dosage forms are retained in the stomach for a longer period of time than smaller ones. Thus, the fed mode is typically induced in a patient by the presence of food in the stomach. The fed mode typically will keep a drug above or at the absoption window for a longer period of time.

In the normal digestive process, the passage of matter through the stomach is delayed by a physiological condition that is variously referred to as the digestive mode, the postprandial mode, or the “fed mode.” Between fed modes, the stomach is in the interdigestive or “fasting” mode. The difference between the two modes lies in the pattern of gastroduodenal motor activity.

As used herein, the term “peak” refers to the plasma drug concentrations reaching the highest point “maxima” either at specified value or time point from the baseline values followed by declining plasma concentrations back to the baseline or below, usually referred to as the minima. Thus, from one minima to the next minima, there is only one peak. In certain embodiments, a peak requires an ascending release rate for at least two hours. The peak may be considered the highest point within a time window of 1, 2, 3, 4, 5 or 6 hours.

As used herein, the phrase “pharmaceutically acceptable,” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained.

As used herein, the term “polymer” refers to a molecule containing a plurality of covalently attached monomer units, and may include branched, dendrimeric and star polymers as well as linear polymers. The term also includes both homopolymers and copolymers, e.g., random copolymers, block copolymers and graft copolymers, as well as uncrosslinked polymers and slightly to moderately to substantially crosslinked polymers. The polymers used in the invention are biocompatible.

As used herein, the term “poorly soluble”, in reference to a drug, refers a drug that is either “substantially water-insoluble,” which means that the drug has an aqueous solubility at any gastrointestinal physiologically relevant pH of less than 0.01 mg/mL, or “sparingly water-soluble,” that is, has an aqueous solubility up to about 1 to 2 mg/mL. The dosage forms of the invention find greater utility as the solubility of the drug decreases. Thus, dosage forms of the present invention are preferred for low-solubility drugs having a solubility of less than 2.0 mg/mL, more preferred for low-solubility drugs having a solubility of less than 1.0 mg/mL, more preferred for low-solubility drugs having a solubility of less than 0.5 mg/mL, and even more preferred for low-solubility drugs having a solubility of less than 0.2 mg/mL at any gastrointestinal physiologically relevant pH.

As used herein, the term “raltegravir” encompasses raltegravir and pharmaceutical acceptable forms and derivatives thereof including salts, esters, amides, prodrugs, active metabolites, analogs and the like. Particularly preferred is the potassium salt of raltegravir. More particularly preferred is the the anhydrous crystalline potassium salt of raltegravir, which is characterized by an X-ray powder diffraction pattern obtained using copper K_{α} radiation which comprises 2Θ values in degrees of 5.9, 20.0 and 20.6. See U.S. Pat. No. 7,754,731, herein incorporated by reference in its entirety.

As used herein, the term “release rate”, in the context of a drug, refers to the quantity of drug released from a dosage form per unit time, e.g., milligrams of drug released per hour (mg/hr). Drug release rates are calculated under in vitro dosage form dissolution testing conditions known in the art. A drug release rate obtained at a specified time “following administration” refers to the in vitro drug release rate obtained at the specified time following implementation of an appropriate dissolution test.

As used herein, the term “subject” (used interchangeably herein with “patient”) refers to an animal, preferably a mammal, most preferably a human, who has been the object of treatment, observation or experiment.

As used herein, the term “swellable”, in the context of polymers, refers to polymers that are capable of absorbing water and result in physical swelling, with the extent to which a polymer can swell being determined by its chemical and physical characteristics, including but not limited to type and plurality of functional groups, molecular weight and degree of crosslinking.

As used herein, a “therapeutically effective amount” of a drug, or a pharmaceutically acceptable salt thereof, refers to a nontoxic but sufficient amount to provide the desired effect, e.g., for raltegravir, the treatment of AIDS.

Monolithic systems, comprised of a single polymer that modulates drug release while providing gastro-retention by a polymer swelling mechanism, generally have limitations relating to balancing drug release and gastro-retention. It is widely expected that as the polymer gradually erodes, the gastro-retentive properties of the dosage form become compromised due to decrease in the size/swellability of the monolithic dosage form.

The controlled release oral dosage forms described herein comprise a therapeutically effective amount of a poorly soluble drug in a multilayer dosage form wherein the dosage form continually delivers the poorly soluble drug over a time period in a range of about 1 up to about 24 hours. In certain embodiments of the invention, the poorly soluble drug is continually delivered over a time period in a range from about 0 up to about 24 hours. Shorter delivery times are also contemplated by the invention.

The swellable polymer swells upon imbibition of water and contact with gastric fluid when reaching the stomach. These swelling layers swell in the presence of water in gastric fluid such that the size of the dosage form is sufficiently increased to provide gastric retention in the stomach of a patient. When the multilayer dosage form is a bilayer, the bilayer is composed of an active layer that serves to provide controlled release of the active agent while the non-active layer aids in gastric retention via flotation, swelling, or other means.

As described in the Examples, gastro-retentive bi-layered systems were formulated to have two separate layers each containing a swellable polymer to modulate drug release and gastro-retention. The average molecular weights of the swellable polymers can be similar, but preferably are different enough from each other to provide different erosion rates. Swellable polymers useful in the preparation of a dosage form of the invention include polymers that are non-toxic and that swell in a dimensionally unrestricted manner upon imbibition of water and hence in gastric fluid.

In one embodiment of the present invention, the active layer comprises a swellable polymer having a molecular weight of approximately 4 million, such as a nonionic, water soluble poly(ethelyene oxide) polymer, e.g., POLYOX™ WSR 301 (The Dow Chemical Company, Midland, Mich.), and is primarily responsible for drug release. In one embodiment of the invention, the non-active layer comprises a swellable polymer having a molecular weight of approximately 5 million, such as a nonionic, water soluble poly(ethelyene oxide) polymer, e.g., POLYOX™ WSR Coagulant (The Dow Chemical Company), and is responsble for aiding gastro-retention.

Swellable polymers in the non-active layer generally include high molecular weight polymers having an average molecular weights of at least 2 million, 2.5 million, 3 million, 4 million, 5 million, or 7 million or more. In certain embodiments, the polymer has a molecular weight or 5 million or more. Preferred polymers include polyalkylene oxides, particularly high molecular weight poly(ethylene oxide)s. Examples of suitable poly(ethylene oxide)s include POLYOX WSR Coagulant and POLYOX™ UCARFLOC Polymer 302 (average molecular weight approximately 5 million); POLYOX™ WSR 303 and POLYOX™ UCARFLOC Polymer 304 (average molecular weight approximately 7 million); POLYOX™ WSR 308 and POLYOX™ UCARFLOC Polymer 309 (average molecular weight approximately 8 million); and POLYOX™ UCARFLOC Polymer 310 (average molecular weight approximately 10 million). Each of these polymers is commercially available from The Dow Chemical Company.

The swellable polymer in the non-active layer will generally represent at least 30%, more than 50%, more than 80 wt. %, more than 85 wt %, more than 90 wt %, more than 95 wt %, more than 98% or more than 99%. with the remainder of the non-active layer composed of one or more inactive additives, such as binders, lubricants, disintegrants, fillers, stabilizers, surfactants, coloring agents, swelling enhancers which can also be disintegrants; acidifers; alkalizers, effervescent agents and the like.

Swellable polymers in the active layer generally include one or more polyalkylene oxides of a lower molecular weight than present in the non-active layer and optionally other hydrophilic polymers, including crosslinked hydrophilic polymers. In certain embodiments of the invention, lower molecular weight polyalkylene oxides have number average molecular weights greater than 2 million, for example, 3 million or 4 million. A preferred range is from 2.5 million to 4 million. Examples of such polymers that are available commercially available include POLYOX™ WSR 301 (average molecular weight of 4 million).

The water-swellable polymers in the active layer can be used individually or in combination. Certain combinations will often provide a more controlled release of the drug than their components when used individually. One example is poly(ethylene oxide) combined with xanthan gum or combination of Poly (ethyelene oxide) polymer with HPMC (hydroxypropylmethyl cellulose or Hypromellose).

In exemplary bilayer tablets of the invention, the active agent will represent approximately 1 wt % to 75 wt %, preferably 2 wt % to 30 wt %, more preferably 5 wt. % to 20 wt. % of the active swellable layer, and will not be incorporated in the non-active swellable layer.

The swellable polymer in the active layer will generally represent less than 70% or less than 50 wt. %, including ranges from 5-35% wt % and 5-25% wt % or 10-35 wt % and 10-25 wt %, with the remainder of the swellable layer composed of additional hydrophilic polymers such as poly(N-vinyl lactams), particularly poly(vinylpyrrolidone) (PVP) (e.g., Povidone); one or more inactive additives, such as binders, lubricants, disintegrants, fillers, stabilizers, surfactants, coloring agents, and the like.

Solubilizers such as the surfactants, sodium lauryl sulphate (SLS) and Poloxamer 188 or PLURONIC® F68 (BASF), up to 10.0% w/w and alkalizers such as sodium carbonate, sodium bi-carbonate up to 20.0% w/w can be used in the active layer to enhance raltegrevir dissolution.

Disintegrants are used to facilitate disintegration of the tablet and also enhance swelling, thereby increasing the erosion rate relative to the dissolution rate, and are generally starches, including cross-linked starches, clays, celluloses, including cross-linked cellulose, sodium croscarmellose and low substituted hydroxypropyl cellulose (L-HPC), algins, gums, crosslinked polymers (e.g., crosslinked polyvinyl pyrrolidone) (for example, Crospovidone) including homopolymer of cross-linked N-vinyl-2-pyrrolidone, ion-exchange resin, combination-sodium starch glycolate, and alginic acid. Preferably, a disintegrant will also act as a swelling enhancer. Preferred disintegrants include croscarmellose sodium at 5-35% w/w, preferably 15-25% w/w. Crospovidone can be used instead of croscarmellose sodium.

Lubricants are used to facilitate tablet manufacture, promoting powder flow and preventing particle capping (i.e., particle breakage) when pressure is relieved. Examples of lubricants include magnesium stearate (in a concentration of from 0.25 wt. % to 3 wt. %, preferably from about 0.5 wt. % to 1.0 wt. %), calcium stearate, stearic acid, sodium stearyl fumarate or hydrogenated vegetable oil (preferably comprised of hydrogenated and refined triglycerides of stearic and palmitic acids at about 1 wt. % to 5 wt. %, most preferably less than about 2 wt. %). A preferred combination is the combination of magnesium stearate and sodium stearyl fumarate (preferably in a 1:1 ratio)

Binders are used to impart cohesive qualities to a tablet, and thus ensure that the tablet remains intact after compression. Binders include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like).

Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol.

Solubility-enhancers, including solubilizers per se, emulsifiers, surfactants and complexing agents (e.g., cyclodextrins), may also be advantageously included in the present formulations.

Stabilizers, as well known in the art, are used to inhibit or retard drug decomposition reactions that include, by way of example, oxidative reactions.

In one example, the active layer may comprise, in addition to raltegravir, for instance: about 5-10 wt. % to about 30 wt. %, preferably about 5 wt. % to about 20 wt. % or 10 wt. % to about 20 wt. % polyalkylene oxide; about 0.25 wt. % to about 3 wt. % magnesium stearate or sodium stearyl fumarate or mixtures thereof; about 2.5 wt. % to about 35 wt. % disintegrant; and about 5 wt. % to about 70 wt. % filler.

The bilayer tablets will generally provide for release of at least 80%, preferably at least 85%, and most preferably at least 90%, of the active agent over a time period in the range of about 2 to 24 hours.

The amount of polymer relative to the drug can vary for the active swellable layer, depending on the drug release rate desired and on the polymer, its molecular weight, and excipients that may be present in the formulation. The amount of polymer will be sufficient however to retain at least about 40% of the drug within the matrix one hour after ingestion (or immersion in the gastric fluid). Preferably, the amount of polymer is such that at least 50% of the drug remains in the matrix one hour after ingestion. More preferably, at least 60%, and most preferably at least 80%, of the drug remains in the matrix one hour after ingestion. In all cases, however, the drug will be substantially all released from the matrix within about 24 hours, after ingestion, and the polymeric matrix will remain substantially intact until all of the drug is released. The term “substantially intact” is used herein to denote a polymeric matrix in which the polymer portion substantially retains its size and shape without deterioration due to becoming solubilized in the gastric fluid or due to breakage into fragments or small particles.

The benefits of this invention will be achieved over a wide range of drug loadings, with the weight ratio of drug to polymer ranging in general from 0.01:99.99 to about 80:20 or as high as 90:10. Preferred loadings (expressed in terms of the weight percent of drug relative to total of drug and polymer) are those within the range of 15% to 80%, more preferably within the range of 30% to 80%, and most preferably in certain cases within the range of about 30% to 70%. For certain applications, however, the benefits will be obtained with drug loadings within the range of 0.01% to 80%, and preferably 15% to 80%.

The dosage forms of the invention may also be formulated as trilayer dosage forms. For example, a trilayer tablet may be prepared with two layers as outlined above and may also include a drug in a quickly dissolving layer on the outer surface of the dosage form for immediate release. This layer is referred to as an “immediate release” or IR layer and its purpose is to provide immediate release into the patient's bloodstream upon ingestion of the dosage form without first requiring the drug to diffuse through a polymeric layer. An optimal dose is one that is high enough to quickly raise the blood concentration of the drug but not high enough to produce any transient overdosing.

The IR layer may contain an additional amount of the poorly soluble drug, one or more different drugs (which do not need to be poorly soluble), or any combination thereof.

In one aspect, the immediate-release portion of the dosage form is either a coating applied or deposited over the entire surface of a bilayered dosage form. Immediate release of the drug from the immediate-release layer is achieved by any of various methods known in the art such as spraying, pan coating, and the like, or the drug can be combined with particles of a binding matrix and compressed over a preformed layer of the inactive layer to form a multi-layered tablet. In either case, the immediate-release coating or layer separates relatively quickly from the remainder of the tablet after ingestion, leaving the remainder intact. One example is the use of a very thin layer or coating which by virtue of its thinness is quickly penetrated by gastric fluid allowing fast leaching of the drug. Another example is by incorporating the drug in a mixture that includes a supporting binder or other inert material that dissolves readily in gastric fluid, releasing the drug as the material dissolves. A third is the use of a supporting binder or other inert material that rapidly disintegrates upon contact with gastric fluid, with both the material and the drug quickly dispersing into the fluid as small particles. Examples of materials that rapidly disintegrate and disperse are lactose and microcrystalline cellulose. An example of a suspending agent and binder is hydroxypropyl methyl cellulose. The IR layer may further comprise magnesium stearate, sodium stearyl fumarate or a mixture thereof, and optionally, a disintegrant.

A film coating may also be included on the outer surface of the dosage form for reasons other than an immediate release dose. The coating may thus serve an aesthetic function or a protective function, or it may make the dosage form easier to swallow or mask the taste of the drug.

The total loading of drug in any dosage form described herein is not critical to this invention and may vary widely, although the choice of loading will affect the release rate and in some cases the release rate profile over time. In most cases, the drug constitutes from about 1% to about 98% by weight of the dosage form. In preferred embodiments, the drug constitutes from about 5% to about 95% by weight of the dosage form, and in the most preferred embodiments, the drug constitutes from about 50% to about 93% by weight of the dosage form.

The dosage forms of the invention will generally provide for gastro-retention of at least 10 hours, at least 12 hours or at least 14 hours. Such retention times are based on administration in a fed state.

The performance of the bi- and tri-layered formulations of the invention can be optimized in terms of the type of pre-dose meal (in terms of fat/calorie content), and the type of the post-dose meal.

The type of meal taken pre-dose with bi- and tri-layered gastro-retentive system is important for optimal performance of the system as it pertains to gastro-retention. The pre-dose meal should be administered 30 minutes prior to dosing (per FDA guidance of food effect studies) but may be administered upto 45 minutes prior to dosing.

For optimal performance, the dosage form needs be taken with at least a medium fat/medium calorie meal (˜20 g of fat content or ≧30% of fat content contributing to the overall caloric content of the meal/˜500-600 kcal) for the dosage form to show optimal retention properties and mean C₂₄ trough concentrations that exceed 100 nM for raltegravir. Other suitable meals include high fat/medium calorie (˜50 g of fat or ≧50% of fat content contributing to the caloric content of the meal/˜500-600 kcal), medium fat/high calorie (˜20 g of fat content/˜800-1000 kcal) and high fat/high calorie meals (30 g/˜800-1000 kcal).

The performance of the bi/tri-layered formulation is optimal when the post-dose meal(s) is taken at least 4 hour post dosing of the first meal as per the FDA guidance of food effect studies. The subsequent meal type (post dose meal) can be a standard lunch/dinner diet. A standard meal intake at least 4 hours post dosing of the first meal results in optimal performance of the dosage form with respect to gastro-retention and favorable C₂₄ trough levels.

Tablets in accordance with this invention can be prepared by conventional techniques, including common tabletting methods. These methods involve mixing, comminution, and fabrication steps commonly practiced by and well known to those skilled in the art of manufacturing drug formulations. Examples of such techniques are:

(1) Direct compression using appropriate punches and dies, such as those available from Elizabeth Carbide Die Company, Inc., McKeesport, Pa., USA. The punches and dies are fitted to a suitable rotary tabletting press, such as the Elizabeth-Hata single-sided Hata Auto Press machine, with either 15, 18 or 22 stations, and available from Elizabeth-Hata International, Inc., North Huntington, Pa., USA;

(2) Injection or compression molding using suitable molds fitted to a compression unit, such as those available from Cincinnati Milacron, Plastics Machinery Division, Batavia, Ohio, USA.;

(3) Granulation such as, but not limited to, fluid bed or high shear granulation or roller compaction, followed by compression; and

(4) Extrusion of a paste into a mold or to an extrudate to be cut into lengths.

When tablets are made by direct compression, the addition of lubricants may be helpful and is sometimes important to promote powder flow and to prevent capping of the tablet (the breaking off of a portion of the tablet) when the pressure is relieved. Useful lubricants are sodium stearyl fumarate, magnesium stearate (in a concentration of from 0.25% to 3% by weight, preferably about 1% or less by weight, in the powder mix) or mixture of sodium stearyl fumarate and magnesium stearate, and hydrogenated vegetable oil (preferably hydrogenated and refined triglycerides of stearic and palmitic acids at about 1% to 5% by weight, most preferably about 2% by weight). Additional excipients may be added to enhance powder flowability, tablet hardness, and tablet friability and to reduce adherence to the die wall.

Drugs suitable for the dosage forms of the invention include poorly soluble drugs having a narrow absorption window, drugs which are ionized within the gastrointestinal tract, and drugs requiring active transport. Gastrointestinally active agents are particularly preferred drugs that can be administered using the present dosage forms. These types of drugs include agents for inhibiting gastric acid secretion, such as H2 receptor antagonists (e.g., cimetidine, ranitidine, famotidine, and nizatidine), H+, K+-ATPase inhibitors (also referred to as “proton pump inhibitors”, such as omeprazole and lansoprazole), and antacids (e.g., calcium carbonate, aluminum hydroxide, and magnesium hydroxide). Also included within this general group are agents for treating infection with Helicobacter pylori (H. pylori), such as metronidazole, timidazole, amoxicillin, clarithromycin, tetracycline, thiamphenicol, and bismuth compounds (e.g., bismuth subcitrate and bismuth subsalicylate). Other gastrointestinally active agents administrable using the present dosage forms include, but are not limited to, pentagastrin, carbenoxolone, sulfated polysaccharides such as sucralfate, prostaglandins such as misoprostol, and muscarinic antagonists such as pirenzepine and telenzepine. Additional agents include antidiarrheal agents, antiemetic agents and prokinetic agents such as ondansetron, granisetron, metoclopramide, chlorpromazine, perphenazine, prochlorperazine, promethazine, thiethylperazine, triflupromazine, domperidone, trimethobenzamide, cisapride, motilin, loperamide, diphenoxylate, and octreotide.

Preferred classes of drugs include, but are not limited to, antihypertensives, antianxiety agents, anticlotting agents, anticonvulsants, blood glucose-lowering agents, decongestants, antihistamines, antitussives, antineoplastics, beta blockers, anti-inflammatories, antipsychotic agents, cognitive enhancers, cholesterol-reducing agents, anti-atherosclerotic agents, antiobesity agents, autoimmune disorder agents, anti-impotence agents, antimicrobial agents (e.g., antibacterial and antifungal agents), hypnotic agents, anti-Parkinsonism agents, anti-Alzheimer's disease agents, antibiotics, anti-depressants, antiviral agents, glycogen phosphorylase inhibitors, diuretics, and cholesteryl ester transfer protein inhibitors.

Anti-microbial agents include, but are not limited to, tetracycline antibiotics and related compounds (chlortetracycline, oxytetracycline, demeclocycline, methacycline, doxycycline, minocycline, rolitetracycline); macrolide antibiotics such as erythromycin, clarithromycin, and azithromycin; streptogramin antibiotics such as quinupristin and dalfopristin; beta-lactam antibiotics, including penicillins (e.g., penicillin G, penicillin VK), antistaphylococcal penicillins (e.g., cloxacillin, dicloxacillin, nafcillin, and oxacillin), extended spectrum penicillins (e.g., aminopenicillins such as ampicillin and amoxicillin, and the antipseudomonal penicillins such as carbenicillin), and cephalosporins (e. g., cefadroxil, cefepime, cephalexin, cefazolin, cefoxitin, cefotetan, cefuroxime, cefotaxime, ceftazidime, and ceftriaxone), and carbapenems such as imipenem, meropenem and aztreonam; aminoglycoside antibiotics such as streptomycin, gentamicin, tobramycin, amikacin, and neomycin; glycopeptide antibiotics such as teicoplanin; sulfonamide antibiotics such as sulfacetamide, sulfabenzamide, sulfadiazine, sulfadoxine, sulfamerazine, sulfamethazine, sulfamethizole, and sulfamethoxazole; quinolone antibiotics such as ciprofloxacin, nalidixic acid, and ofloxacin; anti-mycobacterials such as isoniazid, rifampin, rifabutin, ethambutol, pyrazinamide, ethionamide, aminosalicylic, and cycloserine; systemic antifungal agents such as itraconazole, ketoconazole, fluconazole, and amphotericin B; antiviral agents such as acyclovir, famcicylovir, ganciclovir, idoxuridine, sorivudine, trifluridine, valacyclovir, vidarabine, didanosine, stavudine, zalcitabine, zidovudine, amantadine, interferon alpha, ribavirin and rimantadine; and miscellaneous antimicrobial agents such as chloramphenicol, spectinomycin, polymyxin B (colistin), bacitracin, nitrofurantoin, methenamine mandelate and methenamine hippurate.

Anti-diabetic agents include, but are not limited to, acetohexamide, chlorpropamide, ciglitazone, gliclazide, glipizide, glucagon, glyburide, miglitol, pioglitazone, tolazamide, tolbutamide, triampterine, and troglitazone.

Analgesics include, but are not limited to, non-opioid analgesic agents such as apazone, etodolac, difenpiramide, indomethacin, meclofenamate, mefenamic acid, oxaprozin, phenylbutazone, piroxicam, and tolmetin; and opioid analgesics such as alfentanil, buprenorphine, butorphanol, codeine, drocode, fentanyl, hydrocodone, hydromorphone, levorphanol, meperidine, methadone, morphine, nalbuphine, oxycodone, oxymorphone, pentazocine, propoxyphene, sufentanil, and tramadol.

Anti-inflammatory agents include, but are not limited to, nonsteroidal anti-inflammatory agents, e.g., propionic acid derivatives as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, and fenbufen; apazone; diclofenac; difenpiramide; diflunisal; etodolac; indomethacin; ketorolac; meclofenamate; nabumetone; phenylbutazone; piroxicam; sulindac; and tolmetin; steroidal anti-inflammatory agents e.g., hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, and methylprednisolone.

Anti-convulsant (anti-seizure) agents, include, but are not limited to, azetazolamide, carbamazepine, clonazepam, clorazepate, ethosuximide, ethotoin, felbamate, lamotrigine, mephenytoin, mephobarbital, phenytoin, phenobarbital, primidone, trimethadione, vigabatrin, topiramate, and the benzodiazepines.

CNS and respiratory stimulants include, but are not limited to, the following: xanthines such as caffeine and theophylline; amphetamines such as amphetamine, benzphetamine hydrochloride, dextroamphetamine, dextroamphetamine sulfate, levamphetamine, levamphetamine hydrochloride, methamphetamine, and methamphetamine hydrochloride; and miscellaneous stimulants such as methylphenidate, methylphenidate hydrochloride, modafinil, pemoline, sibutramine, and sibutramine hydrochloride.

Neuroleptic drugs include, but are not limited to, antidepressant drugs, antimanic drugs, and antipsychotic agents, wherein antidepressant drugs include (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors citalopram, fluoxetine, fluvoxamine, paroxetine, sertraline, and venlafaxine, (c) monoamine oxidase inhibitors such as phenelzine, tranylcypromine, and (−)-selegiline, and (d) other, “a typical” antidepressants such as nefazodone, trazodone and venlafaxine, and wherein antimanic and antipsychotic agents include (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, pimozide, quetiapine, risperidone, and sertindole.

Hypnotic agents and sedatives include, but are not limited to, clomethiazole, ethinamate, etomidate, glutethimide, meprobamate, methyprylon, zolpidem, and barbiturates (e.g., amobarbital, apropbarbital, butabarbital, butalbital, mephobarbital, methohexital, pentobarbital, phenobarbital, secobarbital, thiopental).

Anxiolytics and tranquilizers include, but are not limited to, benzodiazepines (e.g., alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, triazolam), buspirone, chlordiazepoxide, and droperidol.

Anticancer agents (antineoplastic agents) include, but are not limited to, paclitaxel, docetaxel, camptothecin and its analogues and derivatives (e.g., 9-aminocamptothecin, 9-nitrocamptothecin, 10-hydroxy-camptothecin, irinotecan, topotecan, 20-O-β-glucopyranosyl camptothecin), taxanes (baccatins, cephalomannine and their derivatives), carboplatin, cisplatin, interferon-α2A, interferon-α2B, interferon-αN3 and other agents of the interferon family, levamisole, altretamine, cladribine, tretinoin, procarbazine, dacarbazine, gemcitabine, mitotane, asparaginase, porfimer, mesna, amifostine, mitotic inhibitors including podophyllotoxin derivatives such as teniposide and etoposide and vinca alkaloids such as vinorelbine, vincristine and vinblastine.

Antihyperlipidemic agents (lipid-lowering agents or “hyperlipidemic” agents) include, but are not limited to, HMG-CoA reductase inhibitors such as atorvastatin, simvastatin, pravastatin, lovastatin and cerivastatin, and other lipid-lowering agents such as clofibrate, fenofibrate, gemfibrozil and tacrine.

Antihypertensive agents include, but are not limited to, amlodipine, benazepril, darodipine, diltiazem, doxazosin, enalapril, eposartan, esmolol, felodipine, fenoldopam, fosinopril, guanabenz, guanadrel, guanethidine, guanfacine, hydralazine, losartan, metyrosine, minoxidil, nicardipine, nifedipine, nisoldipine, phenoxybenzamine, prazosin, quinapril, reserpine, terazosin, and valsartan.

Cardiovascular preparations include, but are not limited to, angiotensin converting enzyme (ACE) inhibitors, cardiac glycosides, calcium channel blockers, beta-blockers, antiarrhythmics, cardioprotective agents, and angiotensin II receptor blocking agents. Examples of the foregoing classes of drugs include the following: ACE inhibitors such as enalapril, 1-carboxymethyl-3-1-carboxy-3-pbenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3S-1H-1-benzazepine-1-acetic acid or 3-(1-ethoxycarbonyl-3-phenyl-(1S)-propylamino)-2,3,4,5-tetrahydro-2-oxo-(3S)-benzazepine-1-acetic acid monohydrochloride; cardiac glycosides such as digoxin and digitoxin; inotropes such as aminone and milrinone; calcium channel blockers such as verapamil, nifedipine, nicardipene, felodipine, isradipine, nimodipine, bepridil, amlodipine and diltiazem; beta-blockers such as atenolol, metoprolol; pindolol, propafenone, propranolol, esmolol, sotalol, timolol, and acebutolol; antiarrhythmics such as moricizine, ibutilide, procainamide, quinidine, disopyramide, lidocaine, phenytoin, tocainide, mexiletine, flecainide, encainide, bretylium and amiodarone; and cardioprotective agents such as dexrazoxane and leucovorin; vasodilators such as nitroglycerin; and angiotensin II receptor blocking agents such as losartan, hydrochlorothiazide, irbesartan, candesartan, telmisartan, eposartan, and valsartan. Examples of other cardiac agents that can be used include: amiodarone, amlodipine, atenolol, bepridil, bisoprolol bretylium, captopril, carvedilol, diltiazem, disopyramide, dofetilide, enalaprilat, enalapril, encainide, esmolol, flecainide, fosinopril, ibutilide, inaminone, irbesartan, lidocaine, lisinopril, losartan, metroprolol, nadolol, nicardipine, nifedipine, procainamide, propafenone, propranolol, quinapril, quinidine, ramipril, trandolapril, and verapamil.

Anti-viral agents include, but are not limited to, the antiherpes agents acyclovir, famciclovir, foscamet, ganciclovir, idoxuridine, sorivudine, trifluridine, valacyclovir, and vidarabine; the antiretroviral agents didanosine, stavudine, zalcitabine, and zidovudine; and other antiviral agents such as amantadine, interferon alpha, ribavirin and rimantadine.

Sex steroids include, but are not limited to, progestogens such as acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone. Also included within this general class are estrogens, e.g.: estradiol (i. e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. Androgenic agents, also included within the general class of sex steroids, are drugs such as the naturally occurring androgens androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, dehydroepiandrosterone (DHEA; also termed “prasterone”), sodium dehydroepiandrosterone sulfate, 4-dihydrotestosterone (DHT; also termed “stanolone”), 5α-dihydrotestosterone, dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol and testosterone; pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters; and pharmaceutically acceptable derivatives of testosterone such as methyl testosterone, testolactone, oxymetholone and fluoxymesterone.

Muscarinic receptor agonists include, but are not limited to, choline esters such as acetylcholine, methacholine, carbachol, bethanechol (carbamylmethylcholine), bethanechol chloride, cholinomimetic natural alkaloids and synthetic analogs thereof, including pilocarpine, muscarine, McN-A-343, and oxotremorine. Muscarinic receptor antagonists include, but are not limited to, belladonna alkaloids or semisynthetic or synthetic analogs thereof, such as atropine, scopolamine, homatropine, homatropine methyl bromide, ipratropium, methantheline, methscopolamine and tiotropium.

Peptide drugs include, but are not limited to, the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP), and vasopressin. Other peptidyl drugs are the cytokines, e. g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage colony-stimulating factor (GM-CSF), macrophage colony-stimulating factor, midkine (MD), and thymopoietin. Still other peptidyl drugs that can be advantageously delivered using the present systems include endorphins (e.g., dermorphin, dynorphin, α-endorphin, β-endorphin, γ-endorphin, σ-endorphin, [Leu 5]enkephalin, [Met5]enkephalin, substance P), kinins (e. g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as α1-antitrypsin, α2-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof.

Diuretics that are water insoluble or are sparingly soluble in water include, but are not limited to, azetazolamide, amiloride, azosemide, bendroflumethiazide, bumetamide, chlorothiazide, chlorthalidone, ethacrynic acid, furosemide, hydrochlorothiazide, metolazone, muzolimine, nesiritide, piretamide, spironolactone, torsemide, triamterine, and tripamide.

Specific examples of compounds include carbamazepine, dapsone, griseofulvin, ibuprofen, nifedipine, phenytoin, valproic acid, ziprasidone, carvedilol, chlorpromazine, cisapride, danazol, diclofinac, diflunisal, furosemide, naproxen, saquinavir, tacrolimus, talinolol, tamoxifen, ketoconazole, itraconazole, mebendazole, mefenamic acid, nicardipine, amprenavir, triamcinolone, betamethasone, glibenclamide, and taxol.

In one embodiment of the invention, the drug is raltegravir or pharmaceutically acceptable salt thereof. Raltegravir (N-(4-fluorobenzyl)-5-hydroxy-1-methyl-2-(1-methyl-1-{[(5-methyl-1,3,4-oxadiazol-2-yl)carbonyl]amino}ethyl)-6-oxo-1,6-dihydropyrimidine-4-carboxamide) belongs to a novel class of anti-retroviral drugs indicated for the treatment of human immunodeficiency virus (HIV-1) known as integrase strand inhibitors. See, e.g., U.S. Pat. No. 7,169,780. More particularly preferred is the the anhydrous crystalline potassium salt of raltegravir, which is characterized by an X-ray powder diffraction pattern obtained using copper K_{α} radiation which comprises 2Θ values in degrees of 5.9, 20.0 and 20.6. See U.S. Pat. No. 7,754,731, herein incorporated by reference in its entirety. This form of raltegravir is the active pharmaceutical ingredient (API) in ISENTRESS® tablets. Raltegravir has solubility of 0.02 mg/ml at pH range of 1-6 and 0.48 mg/ml at pH 6.8.

Commercially available tablets of ISENTRESS® contain 400, 100 or 25 mg of raltegravir in the form of the anhydrous crystalline potassium salt and are approved by the FDA in combination with other anti-retroviral agents for the treatment of HIV infection in adult patients. However, these commercial formulations of ISENTRESS® require twice-daily BID administration due to the short terminal half-life of raltegravir of approximately 9 hours and a shorter α-phase half-life of approximately 1 hour.

Various pharmaceutical formulations that may have application for oral administration of raltegravir in solid dosage form have been disclosed in U.S. Patent Application Publication Nos. 20070292504, 20080118559 and 20100081672, and International Patent Application Publication No. WO 2009/002823.

An effective amount of raltegravir per dosage for the treatment of AIDS is typically about 400-800 mg/tablet administered as two tablets providing a total of about 800-1600 mg of daily administered dose.

The final image for a QD formulation could incorporate the IR component (25-400 mg) combined with the CR GR formulation (400-800 mg) in a single dosage unit for administration of up to 2 units of similar dose strength at a total daily dose of 800-1600 mg to achieve similar exposure as the ISENTRESS® formulation and C₂₄ trough concentrations.

In embodiments of the invention having an immediate release layer, the immediate release layer contains from about 25 mg to about 400 mg of raltegravir. In certain embodiments, the total amount of raltegravir dosed in the IR layer is 100 to 300 mg. In certain embodiments, the IR layer may comprise one or more anti-HIV agents either in addition to raltegravir or substituted for raltegravir.

The present invention also relates to dosage forms comprising poorly soluble drugs, such as raltegravir, that provide a release of the drug at an ascending rate at a time beginning between about 6 hours to about 15 hours, between 7 or 11 hours, or between 8 hours to 10 hours. The present invention also relates to dosage forms comprising a poorly soluble drug, such as raltegravir, that provide a release of raltegravir at an ascending rate at at time point anytime within the foregoing ranges, for example, at 6 hours, 7 hours, 8 hours, 9 hours, 10 hours, 11 hours, 12 hours, 13 hours, 14 hours or 15 hours. This is based on the unexpected discovery that dosage forms comprising raltegravir show a PK profile with two peaks or twin maxima leading to 2 distinct ascending absorption rates. The first peak or hump occurs upon administration of the dosage form or shortly thereafter, e.g., within 0 to 6 hours, or within 0 to 4 hours. It is composed of an ascending release rate followed by a decreasing release rate. At about 8-20 hours post dosing, a second ascending release rate occurs followed by a decreasing release rate. The formulation provides a release of raltegravir at an ascending rate at a time beginning between about 8 hours to about 20 hours, between about 10 hours to 20 hours, between about 12 hours to about 20 hours, between about 14 hours to about 20 hours or between about 16 hours to about 20 hours. The second peak is likely consistent with gastric emptying/disintegration time of the dosage form as is also observed with the scintigraphy studies. This unexpected two peak pharmacokinetic profile maintains a drug concentration above the minimum therapeutic plasma level for an extended period of time suitable for once daily dosing. In certain embodiments, there is an initial ascending absorption rate beginning at about 0 to 4 hours or beginning at about 0 to 6 hours. In certain embodiments, the initial ascending rate occurs in a time period between about 0 to 6 hours, but does not need to last the entire 6 hour period. In certain embodiments, the second ascending rate occurs in a time period between about 8 hours to about 20 hours, but does not last the entire 12 hour period.

In some embodiments, the ascending rate for either the first or second ascending rate can last for at least 1, 2, 3, 4, or 5 hours. In some embodiments, the ascending rate lasts no more than 2, 3, 4, 5, 6, 7, or 8 hours.

Dosage forms of raltegravir composed of monolayers, bilayers and trilayers all provided the unique two peak release profile. Such dosage forms may comprise swellable polymers as described above along with the excipients. In certain embodiments, the swellable polymer has a molecular weight of 2.5 million or higher. In certain embodiments, the swellable polymers has a molecular weight of about 4 million. In certain embodiments, the swellable polymer is a nonionic, water-soluble poly(ethylene oxide) polymer.

The dosage forms of the invention can be used in methods for inhibiting HIV integrase, treating or preventing HIV infection comprising administering a dosage form comprising raltegravir.

In certain embodiments of the invention, dosage forms having raltegravir additionally have one or more other anti-HIV agents. An “anti-HIV agent” is any agent that inhibits HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the dosage forms of this invention may incorporate effective amounts of one or more anti-HIV agents selected from HIV antiviral agents, antiinfectives, or vaccines useful for treating HIV infection or AIDS. Suitable HIV antivirals for use in combination with the raltegravir include, for example, those listed in Table 1.

TABLE 1
Antiviral Agents for Treating HIV infection or AIDS

| | | |
|---|---|---|
| CMX157 | | nRTI |
| efavirenz + emtricitabine + | ATRIPLA ® | nnRTI + |
| tenofovir DF | | nRTI |
| emtricitabine FTC | EMTRIVA ® | nPvTI |
| emtricitabine + tenofovir DF | TRUVADA ® | nRTI |
| emvirine | COACTINON ® | nnRTI |
| enfuvirtide | FUZEON ® | FI |
| enteric coated didanosine | VIDEX EC ® | nRTI |
| 4′-ethynyl-2-fluoro-2′- | | nRTI |
| deoxyadenosine | | |
| etravirine | TMC-125 | nnRTI |
| fosamprenavir calcium | LEXIVA ® | PI |
| indinavir | CRIXIVAN ® | PI |
| lamivudine 3TC | EPIVIR ® | nRTI |
| lamivudine + zidovudine | COMBIVIR ® | nRTI |
| lopinavir | | PI |
| lopinavir + ritonavir | KALETRA ® | PI |
| maraviroc | SELZENTRY ® | EI |
| nelfmavir | VIRACEPT ® | PI |
| nevirapine NVP | VIRAMUNE ® | nnRTI |
| PPL- 100 (also known as PL- | | PI |
| 462) (Ambrilia) | | |
| ritonavir | NORVIR ® | PI |
| saquinavir | INVIRASE ®, | PI |
| | FORTOVASE ® | |
| stavudine, d4T, | ZERIT ® | nRTI |
| didehydrodeoxythymidine | | |
| tenofovir DF (DF = disoproxil | VIREAD ® | nRTI |
| fumarate), TDF | | |
| tipranavir | APTIVUS ® | PI |

EI = entry inhibitor; FI = fusion inhibitor; Inl = integrase inhibitor; PI = protease inhibitor; nRTI = nucleoside reverse transcriptase inhibitor; nnRTI = non-nucleoside reverse transcriptase inhibitor.

Some of the drugs listed in the table are used in a salt form; e.g., abacavir sulfate, indinavir sulfate, atazanavir sulfate, nelfmavir mesylate.

It is understood that the scope of the dosage forms of this invention incorporating other anti-HIV agents is not limited to the HIV antivirals listed in Table 1 and/or listed in the Tables in International Patent Application Nos. WO 01/38332 and WO 02/30930, but includes in principle any combination with any pharmaceutical composition useful for the treatment or prophylaxis of AIDS. The HIV antiviral agents and other agents will typically be employed in these dosage forms in their conventional dosage ranges and regimens as reported in the art, including, for example, the dosages described in the Physicians' Desk Reference, Thomson PDR, Thomson PDR, 57^{th} edition (2003), the 58^{th} edition (2004), or the 59^{th} edition (2005). The dosage ranges for raltegravir in these combination dosage forms are the same as those set forth above.

Dosage forms of the invention comprising raltegravir are useful in the inhibition of HIV integrase, the treatment or prophylaxis of infection by HIV and the treatment, prophylaxis, or the delay in the onset of consequent pathological conditions such as AIDS. Treating AIDS, the prophylaxis of AIDS, delaying the onset of AIDS, treating HIV infection, or prophylaxis of HIV infection is defined as including, but not limited to, treatment or prophylaxis of a wide range of states of HIV infection: AIDS, ARC, both symptomatic and asymptomatic, and actual or potential exposure to HIV. For example, the dosage forms of this invention are useful in the treatment or prophylaxis of infection by HIV after suspected past exposure to HIV by such means as blood transfusion, exchange of body fluids, bites, accidental needle stick, or exposure to patient blood during surgery.

The present invention includes a method for inhibiting HIV integrase (e.g., HIV-1 integrase) in a subject in need thereof which comprises administering to the subject a dosage form as described herein comprising raltegravir. The invention also includes a method for the treatment or prophylaxis of HIV infection (e.g., HIV-1 infection) or for the treatment, prophylaxis, or delay in the onset of AIDS (e.g., AIDS caused by HIV-1) in a subject in need thereof, which comprises administering to the subject a dosage form as described herein. In these methods, the dosage form of the invention can optionally be employed in combination with one or more anti-HIV agents selected from HIV antiviral agents, anti-infective agents, and immunomodulators.

When a dosage form of the present invention is employed or administered in combination with another agent (e.g., an anti-HIV agent), the dosage form and the second agent can be administered separately or together, and when administered separately, the dosage form and second agent can be given concurrently or at different times (e.g., alternately).

The present invention also includes a dosage form comprising raltegravir for oral administration which is a dosage form as described herein (i) for use in, (ii) for use as a medicament for, or (iii) for use in the preparation or manufacture of a medicament for: (a) therapy (e.g., of the human body), (b) medicine, (c) inhibition of HIV integrase, (d) treatment or prophylaxis of infection by HIV, or (e) treatment, prophylaxis of, or delay in the onset or progression of AIDS. In these uses, the dosage forms of the invention can optionally be employed in combination with one or more anti-HIV agents selected from HIV antiviral agents, anti-infective agents, and immunomodulators.

### EXAMPLES

### Example 1

### Preparation of Raltegravir Granules

A high shear wet granulation process was used to manufacture raltegravir potassium salt granules for inclusion in the dosage forms.

In a High Shear Granulator (Aeromatic Fielder PMA 60 from GEA Pharma Systems), the following were charged in the order listed: Raltegravir Potassium (anhydrous crystalline), Hypromellose and Croscarmellose Sodium. The ingredients were dry mixed for 5 minutes at ˜180 RPM and chopper set at low setting (˜2000 rpm).

4250.0 grams of purified water was charged as the granulating fluid to granulate the above dry blend to a satisfactory end point. Water was sprayed at 850 g/min into the granulating bowl over 5.0 minutes with the impellar speed at ˜180 rpm and the chopper speed at low setting. Wet massing followed for 30-90 secs to get to the desired end point of granulation.

The wet granules were passed through a 375Q screen using Quadro co-mill at 1000 rpm and then granules were loaded in a fluid bed dryer (MP2/3 by GEA Pharma Systems) for drying.

The granules were finally dried in a fluid bed dryer (MP-2/3 by GEA Pharma Systems) to a LOD≦2.0% using an inlet temperature of 70° C. The inlet air flow was adjusted in steps as follows: 150 cfm for 15 minutes followed by change to 135 cfm for 10 minutes followed by final change in air flow to 90 cfm until desired LOD was achieved.

The dried granules were milled using Quadro Co-mill using #40G screen at 2000 rpm.

TABLE 2
Composition of Raltegravir High Shear Granules

| | Quantity per | | |
|---|---|---|---|
| | 10.0 kg batch | | |
| Composition | (g) | Quantity | Function |
| Raltegravir, | 8570.0 | 85.7% | active |
| potassium salt | | | |
| hydroxypropyl methyl | 570.0 | 5.7% | binder |
| cellulose (HPMC)^{a} | | | |
| croscarmellose | 860.0 | 8.6% | disintegrant |
| sodium^{b} | | | |
| Purified Water | q.s.^{d} for wet | N/A | granulation fluid |
| | granulation | | |

^{a}supplied as Pharmacoat 606 (Shin-etsu Chemical Co., Tokyo, Japan)
^{b}supplied as AC-DI-SOL ® SD-711 (FMC Biopolymers, Inc., Philadelphia, PA)
c: water is removed during drying process
^{d}q.s = quantum sufficit (sufficient quantity)

### Preparation of Raltegravir Dosage Forms

Two dimensional design space was used for the bilayer formulations with the intent to adjust the polymer content of the non-active layer, such that POLYOX™ WSR Coagulant was 9.7%-23.9% w/w of the total formulation, to optimise the gastroretentive properties of the dosage form, and the polymer content of the active (drug containing) layer, such that POLYOX™ WSR 301 was 10-25% w/w of the active layer, to optimise the drug release rate properties of the tablet.

The dosage forms were made according to the formulations shown in Table 3.

TABLE 3
Composition of Raltegravir Bilayer Formulations

| | | % | % | % | % |
|---|---|---|---|---|---|
| | | w/w | w/w | w/w | w/w |
| | Function | (GR#1 | (GR#2) | (GR#3) | (GR#4) |
| Composition | | | | | |
| (Layer 1) | | | | | |
| Raltegravir | API | 54.81 | 54.81 | 54.81 | 54.81 |
| granules | granules | | | | |
| POLYOX ™ | CR | 25 | 25 | 10 | 10 |
| WSR 301 | Polymer | | | | |
| Sodium | Swelling | 15 | 15 | 15 | 15 |
| Croscarmellose | Enhancer | | | | |
| Lactose | Diluent | 4.19 | 4.19 | 19.19 | 19.19 |
| Monohydrate | | | | | |
| Magnesium | Lubricant | 0.5 | 0.5 | 0.5 | 0.5 |
| Stearate | | | | | |
| Sodium Stearyl | Lubricant | 0.5 | 0.5 | 0.5 | 0.5 |
| Fumarate | | | | | |
| Weight of | | 925 | 925 | 925 | 925 |
| Layer 1 (mg) | | | | | |
| Composition | | | | | |
| (Layer 2) | | | | | |
| POLYOX ™ | CR | 99 | 99 | 99 | 99 |
| WSR | Polymer | (9.7%) | (23.9%) | (9.7%) | (23.9%) |
| Coagulant | | | | | |
| Sodium Stearyl | Lubricant | 1 | 1 | 1 | 1 |
| Fumarate | | | | | |
| Weight of | | 100 | 295 | 100 | 295 |
| Layer 2 (mg) | | | | | |
| Total Tablet | | 1025 | 1220 | 1025 | 1220 |
| Weight (mg) | | | | | |
| Dose Strength | | 400 | 400 | 400 | 400 |
| (mg) | | | | | |

For comparison purposes, a monolithic formulation was prepared according to that described in Table 4. Single dimensional design space flexibility around the polymer content of the monolithic formulation was formulated with the goal of modulating gastroretention and drug release properties from the GR dosage forms by varying the level of polyethylene oxide POLYOX™ WSR 301 NF from 12.5%-32.5% w/w of the tablet weight.

TABLE 4
Composition of Monolithic Raltegravir Formulation

| | | % w/w | % w/w |
|---|---|---|---|
| Composition | Function | (M-1) | (M-2) |
| Raltegravir Granules | API Granules | 50.70 | 36.21 |
| POLYOX ™ WSR 301 | CR Polymer | 12.5 | 32.5 |
| NF | | | |
| Sodium Croscarmellose | Swelling Enhancer | 25 | 25 |
| Lactose Monohydrate | Diluent | 10.8 | 5.29 |
| Magnesium Stearate | Lubricant | 0.5 | 0.5 |
| Sodium Stearyl | Lubricant | 0.5 | 0.5 |
| Fumarate | | | |
| | Dose Strength (mg) | 400 | 400 |
| | Total Tablet weight | 1000 mg | 1400 mg |

Gastro-retentive formulations composed of various polymers especially different M.W. grades of polyethylene oxide (PEO) were evaluated for mapping out their swelling profiles and their specific ability to swell rapidly to avoid premature gastric emptying. The formulations swelled to at least twice its original size within one hour and some of these were determined to swell to ˜200-400% at the t=9 hours using either USP disintegration testing/USP I basket method (#20 mesh) for the swelling evaluation. The dosage form measurements were determined to be at least ˜13 mm in 2 dimensions and in some cases in all 3 dimensions at the end of 9 hours using disintegration testing/USP basket method. The formulations were designed to result in drug release profiles ˜50-90% at t=12 hours to provide flexibility of choosing formulation compositions with different release profiles within the established design space for clinical testing.

Owing to its poor colonic absorption, the GR formulations in Example 1 were evaluated for prolonged retention in the stomach under fed conditions and an ability to deliver the solubilized drug at a controlled rate to the upper GI tract aiming to achieve adequate C₂₄ trough concentrations. Previous efforts to enable QD dosing of raltegravir failed to achieve adequate C₂₄ trough concentrations which included formulation of IR/CR dosage form (conventional CR based matrix formulation) and dosing of the current 400 BID dosing formulation as 800 mg QD.

### Example 2

### Swelling Studies

The gastro-retentive tablets from Example 1 were weighed individually (designated as W0) and placed separately in a dissolution bath using a bolus basket (Distek Inc, NJ Model-2100C) or a disintegration apparatus (Vankel, NJ Model-VK-100) containing 900 ml of 0.1 N HCl (Fischer Scientific) or distilled water or FeSSIF media (Biorelevant.com, Croyden, U.K) and incubated at 37° C.±1° C. at 100 rpm paddle speed. At regular time intervals until 9 hours, the tablets were removed from the beaker, and the excess surface liquid was removed carefully using tissue paper. The swollen floating tablets were then re-weighed (Wt), and % swelling was calculated using the following formula below.

% Swelling at time “t”=(Wt−W0/W0)×100 where W0 is the initial tablet weight

The dimensions of the tablets were also measured using a vernier caliper to determine the length, breadth and the thickness of the tablets.

Swelling data was obtained for the two extreme ends of the design space for the bi-layered tablets and the monolithic tablets using either USP bolus basket method or USP disintegration apparatus taking tablets at one hour time intervals to measure % swelling.

These swellability tests show that not only do these monolithic and bi-layerd GR systems swell to 3-4 times their original size but they swell to at least double its original size within an hour of immersing them in the an aqueous based media.

These in vitro tests demonstrate that the gastro-retentive dosage forms are not prone to empty prematurely from the stomach and should be retained in the stomach for an extended period of time.

### Example 3

### Radioimaging of Raltegravir Formulations

In order to definitively show that the GR formulations have a prolonged retention time in the stomach and upper GI tract, radiolabelled raltegravir was visualized using anterior scintigraphic images.

An ion-exchange resin was used which has ¹¹¹In radiolabel. The radiolabelled resin was added to the active blend prior to tablet compression. Eight subjects were administered radiolabeled doses not more than 0.05 MBq ¹¹¹In contained in the tablet as part of the active layer.

In vivo gamma scintigraphic imaging was performed as follows:

An anterior anatomical marker containing not more than 0.05 MBq ¹¹¹In was taped to the skin where the mid-clavicular line meets with the right costal margin so that it lies in approximately the same transverse plane as the pylorus.

Anterior scintigraphic images, each of at least 50 seconds duration, were recorded using a gamma camera (General Electric Maxicamera) with a 40 cm field of view (FOV) and fitted with a low energy general all-purpose parallel hole collimator. Image duration was increased as necessary to ensure the quality of the data. All images were recorded at approximately 30 min intervals until 12 hours post-dose, and then every hour until 16 hour post-dose. A final image was acquired at 24 hour post-dose.

Results shown in Table 5 demonstrate a positive proof of concept with a bi-layered GR formulation resulting in prolonged gastro-retention of up to at least ˜14-16 hours with complete disintegration of the dosage forms occurring at ˜17 hours in the stomach/small intestine in the fed state. The bi-layered formulation also showed substantially improved gastric retention times over a monolithic dosage form.

TABLE 5
Gastric Retention Times of Raltegravir Formulations

| | Time last observed in | Time for complete |
|---|---|---|
| | stomach (hours post dose) | time of disintegration |
| GR#2 | | |
| (Bi-layered) | | |
| Mean | 14.645 | 17.363 |
| SD | 1.888 | 3.505 |
| Median | 15.990 | 19.840 |
| n | 8 | 8 |
| GR#5 | | |
| (Monolithics) | | |
| Mean | 12.129 | 13.353 |
| SD | 2.560 | 4.133 |
| Median | 11.500 | 11.770 |
| n | 8 | 8 |

### Example 4

### Pharmacokinetic Studies of Raltegravir Formulations

Owing to its poor colonic absorption, one design goal of the formulations of the invention was to deliver the solubilized drug at a controlled rate to the upper GI tract aiming to achieve efficacious C₂₄ trough concentrations. Previous efforts to enable QD dosing of raltegravir failed to achieve the desired trough concentrations which included formulation of IR/CR dosage form (conventional CR based matrix formulation) and dosing of the current 400 BID dosing formulation as 800 mg QD.

A pharmacokinetic and scintigraphic study was performed to investigate the performance of novel controlled release (gastroretentive) formulations of raltegravir.

For each of the prototype CR formulations and meal content, 8 subjects received a radiolabelled formulation and had scintigraphic images acquired. The same 8 subjects received each radiolabelled CR formulation. The commercial formulation containing poloxamer was not radiolabelled.

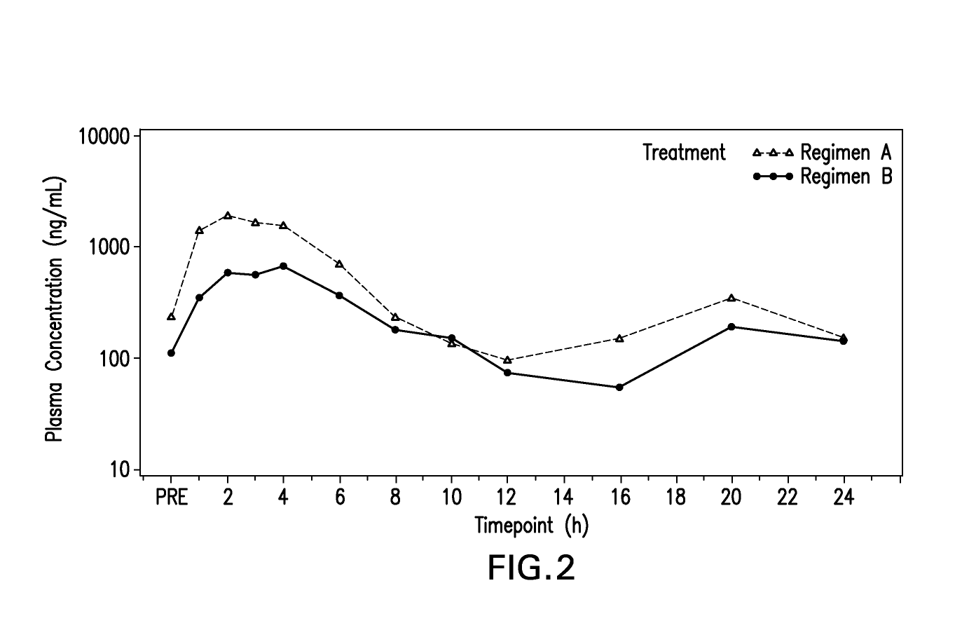

All subjects had serial blood sampling performed for the analysis of raltegravir plasma concentrations. PK blood samples were withdrawn at regular intervals at pre-dose and up to 48 hours post-dose. Non-compartmental analysis of the PK data was performed using industry standard software (WinNonlin version 5.1 Pharsight®, USA). Pharmacokinetic parameters of raltegravir after a single administration of the prototype controlled release (CR) formulations of raltegravir were estimated, and the plasma concentration of raltegravir were compared to the plasma concentration at 12 hours (C₁₂) following administration of a single ISENTRESS® tablet. Results are provided in Table 6 and FIG. 2A.

TABLE 6
Summary Statistics (GM and GM CV %) of Raltegravir
Plasma Pharmacokinetic Parameters Following Administration
of a Single Oral Dose in Healthy Volunteers (median
(min, max) for Tmax)

| | Mean C12 hr | Mean C24 hr |
|---|---|---|
| Treatment | (ng/mL) | (ng/mL) |
| ISENTRESS ® (n = 16) | 153 | 9.18 |
| 400 mg (Period 1) | | |

Gastroretentive Formulations

| GR#1 (n = 16) | 89.6 | 59.6 |
|---|---|---|
| 400 mg (Period 2) | | |
| GMR (GR#1/ISENTRESS ®) | 0.58 | 6.49 |
| GMR | 0.39 (Range 0.02-3.94) | |
| (GR#1 C24 hr/ISENTRESS ® C12 hr) | | |
| GR#2 (n = 15) | 121 | 82.6 |
| 400 mg (Period 3) | | |
| GMR (GR#2/ISENTRESS ®) | 0.81 | 9.26 |
| GMR | 0.56 (Range 0.05-7.36) | |
| (GR#2 C24 hr/ISENTRESS ® C12 hr) | | |
| GR#3 (n = 9) | 115 | 49.5 |
| 400 mg (Period 4) | | |
| GMR (GR#3/ISENTRESS ®) | 0.84 | 6.61 |
| GMR | 0.68 (Range 0.08-2.68) | |
| (GR#3 C24 hr/ISENTRESS ® C12 hr) | | |
| GR#4 (n = 11) | 393 | 42.2 |
| 400 mg (Period 5) | | |
| GMR (GR#4/ISENTRESS ®) | 2.46 | 4.95 |
| GMR | 0.26 (Range 0.01-2.76) | |
| (GR#4 C24 hr/ISENTRESS ® C12 hr) | | |
| GR#2 (n = 11) | 780 | 91.3 |
| 800 mg (Period 6) | | |
| GMR (GR#2/ISENTRESS ®) | 5.36 | 11.34 |

^{†}Median (Min-Max)

At 24 hours post-dose, each of the GR formulations showed a significant increase in C₂₄ hr compared to ISENTRESS® (all greater than five-fold).

Bilayer formulations GR#1 through GR#3 showed similar pharmacokinetics, indicating that the differential release rates tested in this study did not have a substantial effect on the pharmacokinetics of raltegravir.

A unique two peak profile was observed for the monolayer, bilayered and trilayered dosage forms.

The results of this study indicate that raltegravir C₂₄ hr concentrations can be substantially increased by the GR formulations described by this invention.

### Example 5

### Composition of Raltegravir IR/GR Trilayered Prototype Tablets (Prototypes 1, 2, 3 and 4)

The components and quantitative composition of raltegravir IR/GR Trilayered Prototype Tablet Formulations are given in Table 6. In line with the formulation design space approach, these formulations represent the extremes of concentrations of dose that could be used in the study (e.g., 1265.00 mg to 1480.00 mg).

A wet granulation process was used to manufacture Raltegravir High Shear Granules as described in Example 1. This granulate was used to manufacture a trilayered tablet, where for two of the layers, the granules were combined with other excipients to create a blend which is subsequently compressed into tablets, whilst the third layer was a polymer layer (for swelling purposes) containing only excipients. The CR blend was prepared by blending the active raltegravir (RAL) granules with croscarmellose sodium (sieved) and POLYOX™ WSR 301 (sieved); followed by blending with the lubricants Mg stearate and sodium stearyl fumarate (sieved).

The neat polymer layer was prepared by blending polyethylene oxide (POLYOX™ WSR Coagulant) with the lubricant like sodium stearyl fumarate (sieved).

The IR blend was prepared by blending the active raltegravir granules with microcrystalline cellulose, and optionally a surfactant such as Poloxamer 188, and lubricants such as magnesium stearate (sieved) and sodium stearyl fumarate (sieved)

The tablets were manufactured using a Hata Tri-layered press HT-CVX-45 station press. Briefly, the tablets were manufactured by applying a tamping force on the 1^{st} layer (active CR layer) followed by application of tamping force on Layer 2 (inactive drug layer) followed by application of pre-compression and main compression force on the final tablets after addition of the 3^{rd} layer (IR) layer to make tri-layered tablets (oval shaped tooling). These tri-layered tablets were finally film coated using Opadry II with up to ˜2.5-3.0% weight gain.

Examples of trilayer formulations are shown in Table 7.

TABLE 7
Raltegravir Trilayered Formulations

| | % w/w | mg/tab | Total Dose/tab |
|---|---|---|---|
| Layer 1 (CR layer) | | | |
| Ral Granules | 55.41 | 507.0 | 400 mg |
| POLYOX ™ WSR 301 | 15.00 | 137.25 | |
| Croscarmelose Na | 15.00 | 137.25 | |
| AVICEL ® PH 102 | 13.59 | 124.35 | |
| Mag stearate | 0.50 | 4.6 | |
| SSF | 0.50 | 4.6 | |
| Layer 1 weight | 100.00 | 915.0 | |
| Layer 2 | | | |
| (Inactive Layer) | | | |
| POLYOX ™ Coag | 67 | 134.0 | |
| AVICEL ® PH 102 | 32.5 | 65.0 | |
| SSF | 0.5 | 1.0 | |
| | 100 | 200.0 | |
| Layer 3 (IR layer) | | | |
| Ral Granules | 42.3 | 63.4 | 50 mg |
| AVICEL ® PH102 | 56.7 | 85.1 | |
| Sodium stearyl | 0.50 | 0.75 | |
| fumarate | | | |
| Mag stearate | 0.50 | 0.75 | |
| Layer 3 weight | 100 | 150 | |
| Total Tablet Weight | | 1265 | |
| Total Dose | | | 450 mg |
| Layer 1 (CR layer) | | | |
| Ral Granules | 71.08 | 760.5 | 600 mg |
| POLYOX ™ WSR 301 | 12.85 | 137.5 | |
| Croscarmelose Na | 15.07 | 161.2 | |
| Mag stearate | 0.50 | 5.4 | |
| SSF | 0.50 | 5.4 | |
| Layer 1 weight | 100.00 | 1070 | |
| Layer 2 | | | |
| (Inactive Layer) | | | |
| POLYOX ™ Coag | 67 | 134.0 | |
| AVICEL ® PH 102 | 32.50 | 65.0 | |
| SSF | 0.5 | 1.0 | |
| | 100 | 200.0 | |
| Layer 3 (IR layer) | | | |
| Ral Granules | 42.3 | 63.4 | 50 mg |
| AVICEL ® PH102 | 56.7 | 85.1 | |
| Sodium stearyl | 0.50 | 0.75 | |
| fumarate | | | |
| Mag stearate | 0.50 | 0.75 | |
| Layer 3 weight | 100 | 150 | |
| Total Tablet Weight | | 1420 | |
| Total Dose | | | 650 mg |
| Layer 1 (CR layer) | | | |
| Ral Granules | 55.41 | 507.0 | 400 mg |
| POLYOX ™ WSR 301 | 15.00 | 137.25 | |
| Croscarmelose Na | 15.00 | 137.25 | |
| AVICEL ® PH 102 | 13.59 | 124.35 | |
| Mag stearate | 0.50 | 4.6 | |
| SSF | 0.50 | 4.6 | |
| Layer 1 weight | 100 | 915 | |
| Layer 2 | | | |
| (Inactive Layer) | | | |
| POLYOX ™ WSR | 67 | 134.0 | |
| Coag | | | |
| AVICEL ® PH 102 | 32.5 | 65.0 | |
| SSF | 0.5 | 1.0 | |
| | 100 | 200.0 | |
| Layer 3 (IR layer) | | | |
| Ral Granules | 90.5 | 190.1 | 150 mg |
| AVICEL ® PH102 | 8.5 | 17.9 | |
| Sodium stearyl | 0.50 | 1.05 | |
| fumarate | | | |
| Mag stearate | 0.50 | 1.05 | |
| Layer 3 weight | 100 | 210 | |
| Total Tablet Weight | | 1325 | |
| Total Dose | | | 550 mg |
| Layer 1 (CR layer) | | | |
| Ral Granules | 71.08 | 760.5 | 600 mg |
| POLYOX ™ WSR 301 | 12.85 | 137.5 | |
| Croscarmelose Na | 15.07 | 161.2 | |
| Mag stearate | 0.50 | 5.4 | |
| SSF | 0.50 | 5.4 | |
| Layer 1 weight | 100.00 | 1070 | |
| Layer 2 | | | |
| (Inactive Layer) | | | |
| POLYOX ™ WSR | 67 | 134.0 | |
| Coag | | | |
| AVICEL ® PH 102 | 32.50 | 65.0 | |
| SSF | 0.5 | 1.0 | |
| | 100 | 200.0 | |
| Layer 3 (IR layer) | | | 150 mg |
| Ral Granules | 90.5 | 190.1 | |
| AVICEL ® PH102 | 8.5 | 17.9 | |
| Sodium stearyl | 0.50 | 1.05 | |
| fumarate | | | |
| Mag stearate | 0.50 | 1.05 | |
| Layer 3 weight | 100 | 210.0 | |
| Total Tablet Weight | | 1480 | |
| Total Dose | | | 750 mg |
| Layer 1 (CR layer) | | | |
| Ral Granules | 71.08 | 760.5 | 600 mg |
| POLYOX ™ WSR 301 | 12.85 | 137.5 | |
| Croscarmelose Na | 15.07 | 161.2 | |
| Mag stearate | 0.50 | 5.4 | |
| SSF | 0.50 | 5.4 | |
| Layer 1 weight | 100.00 | 1070 | |
| Layer 2 | | | |
| (Inactive Layer) | | | |
| POLYOX ™ WSR Coag | 67 | 134.0 | |
| AVICEL ® PH 102 | 32.50 | 65.0 | |
| SSF | 0.5 | 1.0 | |
| | 100 | 200.0 | |
| Layer 3 (IR layer) | | | 150 mg |
| Ral Granules | 82.6 | 190.05 | |
| AVICEL ® PH102 | 7.7 | 17.65 | |
| Poloxamer 188 | 8.7 | 20.0 | |
| Sodium stearyl | 0.50 | 1.15 | |
| fumarate | | | |
| Mag stearate | 0.50 | 1.15 | |
| Layer 3 weight | 100 | 230.0 | |
| Total Tablet Weight | | 1500 | |
| Total Dose | | | 750 mg |

### Example 6

### Raltegravir IR/GR Trilayered Prototype Tablet Pharmacokinetic Study

Two tablets of the Raltegravir IR/GR Trilayered Prototype #4 (600 mg Raltegravir in the CR layer/150 mg Raltegravir in the IR layer) were orally administered to 20 healthy human subjects following a standard high fat meal. Pharmacokinetic studies were performed as described in Example 4.

Exposure to 1500 mg Raltegravir IR/CR trilayer tablet with a high fat meal resulted in a relatively flat pharmacokinetic profile with geometric mean C₂₄ hr values above minimum required therapeutic plasma concentrations. Results are shown in FIG. 2B demonstrating that gastroretention and resulting pharmacokinetics of raltegravir maintained a substantial plasma concentration for more than 24 hours.

### Example 7

Regimen A: 1500 mg Raltegravir as 2×150/600 mg IR/GR trilayer tablets administered orally at lunchtime with a moderate fat/moderate calorie lunch followed by a standard dinner.
Regimen B: 1500 mg Raltegravir as 2×150/600 mg IR/GR trilayer tablets administered orally in the morning with a moderate fat/moderate calorie breakfast followed by a standard lunch.

Exposure to 1500 mg Raltegravir IR/CR trilayer tablet with a medium fat/medium calorie diet breakfast or lunch followed by a standard meal at least 4 hours post dosing resulted in a relatively flat pharmacokinetic profile with geometric mean C24 hr values above minimum required therapeutic plasma concentrations. Results are shown in Table 8 demonstrating that gastroretention and resulting pharmacokinetics of raltegravir maintained a subtantial plasma concentration for more than 24 hours.

TABLE 8
Geometric Mean (GM) of Pharmacokinetic Parameter
Values for Raltegravir Following Administration of
Single 1500 mg Oral Doses of IR/GR Raltegravir (2 ×
150/600 mg tablets) to Healthy Subjects

| | | | C24 hr | AUC₀₋ₗₐₛₜ | Cmax | Tmax |
|---|---|---|---|---|---|---|
| Regimen | Description | N | (nM) | (μM-hr) | (μM) | (hr)^{a} |
| (A)Tri- | Lunch dosing | 10 | 119.0 | 26.3 | 3.4 | 4.0 |
| Layered | MF/MC lunch | | | | | |
| | Standard dinner | | | | | |
| (B) Tri- | Breakfast dosing | 10 | 158.0 | 18.0 | 3.0 | 3.0 |
| layered | MF/MC breakfast | | | | | |
| | Standard lunch | | | | | |

Regimen C: 1500 mg Raltegravir as 2×150/600 mg IR/GR trilayer tablets administered orally with a high fat/high calorie meal followed by a standard meal at least 4 hrs post dosing.
Regimen D: 1500 mg Raltegravir as 2×150/600 mg IR/GR trilayer tablets administered orally with a moderate fat/moderate calorie meal followed by a standard meal at least 4 hrs post dosing.
Regimen E: 1500 mg Raltegravir as 2×150/600 mg IR/GR trilayer tablets administered orally with a low fat/low calorie meal followed by a standard meal at least 4 hrs post dosing.

Exposure to 1500 mg Raltegravir IR/CR trilayer tablet with a high fat/high calorie or a medium fat/medium calorie meal resulted in a relatively flat pharmacokinetic profile with geometric mean C₂₄ hr values above minimum required therapeutic plasma concentrations. Results are shown in Table 9 demonstrating thatgastroretention and resulting pharmacokinetics of raltegravir maintained a substantial plasma concentration for more than 24 hours. However, administration with a low fat/low calorie diet did not result in favorable PK and trough concentrations.

TABLE 9
Geometric Mean (GM) of Pharmacokinetic Parameter
Values for Raltegravir Following Administration of
Single 1500 mg Oral Doses of IR/GR Raltegravir (2 ×
150/600 mg tablets) to Healthy Subjects

| | Irrespective of food | | | | |
|---|---|---|---|---|---|
| | and time of dosing | | High-fat/ | Mod-fat/ | Low-fat/ |
| | Isent. | Isent. | HC (Reg- | MC (Reg- | LC (Reg- |
| Param- | BID | QD | imen C) | imen D) | imen E) |
| eter | 400 mg | 800 mg | 1500 mg | 1500 mg | |
| AUC0-24 | 26.3 | 30.9 | 38.0 | 31.5 | 10.2 |
| (uM*h) | | | | | |
| Cmax | 3.4 | 13.5 | 3.9 | 3.0 | 1.9 |
| (uM) | | | | | |
| Ctrough | 257 | 40 | 419 | 252 | 48 |
| (nM)^{‡} | | | | | |

While the foregoing specification teaches the principles of the present invention, with examples provided for the purpose of illustration, the practice of the invention encompasses all of the usual variations, adaptations and/or modifications that come within the scope of the following claims.

1. An oral drug dosage form comprising
(a) an active layer comprising a poorly soluble drug having a narrow absorption window, or a pharmaceutically active salt thereof, and a first swellable polymer with an average molecular weight in range from greater than 2 million to 5 million; and
(b) a non-active layer comprising a second swellable polymer with an average molecular weight greater than 4 million
wherein the average molecular weight of the first swellable polymer is equal to or less than the average molecular weight of the second polymer.

2. (canceled)

3. (canceled)

4. The dosage form of claim 1, wherein the first swellable polymer has an average molecular weight in a range from 2.5 million to 4 million and the second polymer has an average molecular weight in a range from 5 million to 10 million.

5. (canceled)

6. The dosage form of claim 1, wherein the first swellable polymer is a nonionic, water soluble poly(ethelyene oxide) polymer that is present in the active layer at a concentration range from 5 to 35% inclusive and the second polymer is a nonionic, water soluble poly(ethelyene oxide) polymer that is present in the non-active layer at a concentration greater than about 30% w/w.

7. (canceled)

8. (canceled)

9. (canceled)

10. (canceled)

11. (canceled)

12. (canceled)

13. The dosage form of claim 1, wherein one or more layers further comprise a lubricant, disintegrant, filler, surfactant, or any combination thereof, wherein: (i) the lubricant is magnesium stearate, calcium stearate, stearic acid, sodium stearyl fumarate or a mixture thereof; (ii) disintegrant is croscarmellose or crospovidone; (iii) the filler is microcrystalline cellulose or lactose; and (iv) wherein the surfactant is poloxamer 188 (Pluronic F68) or sodium lauryl sulfate.

14. (canceled)

15. (canceled)

16. (canceled)

17. (canceled)

18. The dosage form of claim 1, wherein the dosage form is retained in the stomach and upper gastrointestinal tract for at least 10 hours when administered to a subject in the fed state.

19. The dosage form of claim 1, wherein the dosage form is comprised of a tablet.

20. (canceled)

21. The dosage form of claim 1, wherein the poorly soluble drug is an integrase strand transfer inhibitor.

22. (canceled)

23. The dosage form of claim 1 comprising
(a) an active layer comprising a poorly soluble drug having a narrow absorption window, or a pharmaceutically active salt thereof, and a first swellable polymer with an average molecular weight in range from greater than 2 million to 5 million;
(b) a non-active layer comprising a second swellable polymer with a average molecular weight greater than 4 million; and
(c) an immediate release layer comprising the poorly soluble drug, one or more additional drugs, or a combination thereof or pharmaceutically acceptable salts thereof, and a filler that provides for immediate release of the poorly soluble drug and the one or more additional drugs,
wherein the average molecular weight of the first swellable polymer is equal to or less than the average molecular weight of the second polymer.

24. The dosage form of claim 23, wherein the filler comprises cellulose or lactose and wherein said dosage form further comprises magnesium stearate, sodium stearyl fumarate or a mixture thereof.

25. (canceled)

26. The dosage form of claim 23, wherein the second swellable polymer is present in the non-active layer at a concentration greater than about 50% w/w.

27. The dosage form of claim 23, wherein the poorly soluble drug is raltegravir or raltegravir potassium.

28. The dosage form of claim 27 further comprising at least one other anti-HIV agent.

29. (canceled)

30. A method for inhibiting HIV integrase in a subject in need of such inhibition which comprises administering the dosage form of claim 27.

31. A method for the treatment of HIV infection or the treatment, or delay in the onset of AIDS in a subject in need thereof which comprises administering the dosage form of claim 27.

32. (canceled)

33. A dosage form comprising raltegravir that provides an initial peak within 6 hours post dose and a second peak within 8-24 hours post dose when administered to a subject in a fed state.

34. The dosage form of claim 33 which provides an initial ascending rate in a time period from about 0 to about 6 hours when administered to a subject in a fed state.

35. The dosage form of claim 34, wherein the initial ascending rate is maintained for at least 2 hours when administered to a subject in a fed state.

36. The dosage form of claim 33 which provides the second ascending rate in a time period from about 8 to about 20 hours when administered to a subject in a fed state.

37. The dosage form of claim 36 wherein the second ascending rate is maintained for at least 2 hours when administered to a subject in a fed state.

38. (canceled)

39. (canceled)

40. (canceled)

41. A method for treating or preventing HIV infection comprising administering to a subject in a fed state a dosage form comprising raltegravir that provides a release of raltegravir with an initial peak within 6 hours post dose and a second peak within 15-24 hours post dose.

42. (canceled)

43. (canceled)

44. (canceled)

45. (canceled)

46. (canceled)
